(19) United States
(12) Patent Application Publication
(10) Pub. No.: US 2016/0175378 A1
(43) Pub. Date: Jun. 23, 2016

(54) NEW KETOGENIC DIET AND ITS USE IN TREATING THE CRITICALLY ILL

(71) Applicants: Beth Israel Deaconess Medical Center, Boston, MA (US); Children's Medical Center Corporation, Boston, MA (US)

(72) Inventors: Bruce R. Bistrain, Ipswich, MA (US); Mark Puder, Medfield, MA (US)

(21) Appl. No.: 15/056,473

(22) Filed: Feb. 29, 2016

Related U.S. Application Data
(63) Continuation of application No. PCT/US2014/053663, filed on Sep. 2, 2014.
(60) Provisional application No. 61/873,551, filed on Sep. 4, 2013.

(51) Int. Cl. A61K 38/01; A61K 31/202; A61K 31/201

(52) U.S. Cl. CPC A61K 38/011; A61K 31/201; A61K 31/202; A23L 1/3006; A23L 1/305; A23L 1/293; A23L 1/296; A23V 2002/00

(57) ABSTRACT

The present invention provides a novel diet for the critically ill, a ketogenic diet. Many of the problems found in the critically ill, such as hyperglycemia, excessive inflammatory response, and fluid retention, can be traced to the normal high carbohydrate diet used in hospital treatment. By replacing the high carbohydrate diet with a diet low in carbohydrates and higher in the proper fats, many of these problems are ameliorated. The preferred fats have high omega-3 content or are MCT's.

### CROSS REFERENCE TO RELATED APPLICATIONS

This application is a Continuation of International PCT Patent Application No. PCT/US2014/053663, filed Sep. 2, 2014, which application claims the benefit of priority of U.S. Provisional Patent Application No. 61/873,551, filed Sep. 4, 2013, the contents of which are incorporated herein by reference in their entireties.

### BACKGROUND

Treatment of the critically ill provide special problems. Nutritional support is necessary and feeding improves outcome in relation to both morbidity and mortality. However, the diets used to treat the critically ill can cause problems. Glucose has been considered the optimal food for the critically ill because when glucose is metabolized , it requires approximately 8% less oxygen to produce a similar amount of ATP as compared to metabolism of fat. In addition, while providing full or nearly full nutritional support is essential for the critically ill, many patients cannot tolerate standard enteral formulas because of gastrointestinal intolerance or other factors. Full nutritional support with carbohydrate-based regimens for the critically ill can lead to three pathophysiologic complications: hyperglycemia, volume overload, and excessive oxidative stress. The glucose level in the diets used for the critically ill can exacerbate these conditions.

Hyperglycemia often develops in critical illness because the systematic inflammatory response increases the rate of glucose production by up to 50%. It also makes gluconeogenesis either less responsive or completely unresponsive to the provision of exogenous glucose, while limiting peripheral glucose intake due to insulin resistance. Thus, a nutritional formula which provides carbohydrate such as glucose as the principal energy source can lead to hyperglycemia through increased production and exogenous supply with reduced uptake by muscle which is the major tissue for glucose clearance. Hyperglycemia itself causes many problems including the increased risk of nosocomial infection.

Volume overload often occurs because substantial fluid is administered to provide the high carbohydrate diet. While glucose is an efficient energy source in ATP production, it has a relatively low caloric content compared to fat. High levels of fluid are needed to provide sufficient glucose to provide energy support, particularly when using peripheral veins. This is counterproductive because critically ill patients typically have a reduced ability to excrete a fluid load due to organ dysfunction and hormonal changes resulting from the systemic inflammatory response. There are often increases in the level of the antidiuretic hormone aldosterone, and insulin, which may have increased levels due to increased blood glucose levels, can also have antidiuretic properties. These changers foster excessive sodium and water retention, making an overall modification in electrolyte balance, and can lead to a risk of increased morbidity and mortality, since severe fluid retention impairs organ function, particularly of the lungs.

The critically ill may also have an intensification of the systemic inflammatory response by a carbohydrate-rich diet. A diet of this type may lead to an increase in production of reactive oxygen species that, while serving as potent bactericidal agents, can also damage normal tissue and impair function. Hyperglycemia increases cytokine production and an increase in reactive oxygen species. There is also a marked stimulation of de novo lipogenesis which is the endogenous production of triglyceride(fat). De novo lipogenesis and gluconeogenesis are pathways of reductive biosynthesis, which lower the cytosolic and mitochondrial redox states. Thus, a dietary regime based primarily on carbohydrate increases both the production of reactive oxygen species from hyperglycemia and reduces their elimination by accelerating processes of reductive biosynthesis, exacerbating oxidative stress.

The Food and Nutrition Board of the Institute of Medicine sets the Acceptable Macronutrient Distribution Ranges (AMDR) for standard diets as having 20-35% fat, 10-25% protein and 45-65% carbohydrate (all values by calorie content). Most enteral and parenteral nutrition diets meet these standards. However, there has been no controlled testing of whether this type of diet is optimal for the critically ill.

Not all diets used in treatment of various medical conditions meet the AMDR standards.

There is a history of using ketogenic diets for specific purposes for non-critically ill patients. For example, ketogenic diets were and are increasingly used today for treating intractable epilepsy, primarily in infants and children This therapy, although generally successful, fell out of favor when anticonvulsant drugs became readily available, but more recently a resurgence of use has occurred in cases of epilepsy resistant to drugs. The classic ketogenic diet has 3-4 g of fat per gram of protein and carbohydrate combined. However, long term compliance was difficult with such a diet. Accordingly, some ketogenic diets have been modified to improve compliance, for example with the use of medium chain triglycerides (MCT) which permit the greater use of dietary carbohydrate without eliminating ketogenesis; and other changes have led to diets like the modified Atkins diet, which allows a fat to protein plus carbohydrate ratio of about 1 to 1. Despite changing these ratios, all the diets produced ketosis, because ketosis is largely based on the degree of carbohydrate restriction, with a lower threshold limit of about 65 g per day. The principal factor in governing ketosis is a level of insulin, and with low insulin levels, the higher levels of free fatty acids and glucagon foster greater ketogenesis. The ketogenic diet has also been used in the treatment of certain glycolysis-dependent cancers. However, ketogenic diets have not been used for the critically ill because conventional wisdom is that a ketogenic diet would be contraindicated for critically ill patients.

### SUMMARY

The present invention relates to a new form of ketogenic diet and the use of such a diet for treating critically ill patients. The diet is antithetical to the conventional teaching that carbohydrates, primarily simple sugars such as glucose, should be the primary caloric source for the critically ill because glucose requires less oxygen for generating the same amount of ATP than fat.

However, the use of carbohydrate as the primary energy source in the critically ill leads to several attendant problems that can be ameliorated with the present ketogenic diet.

The diet of the present invention has very low carbohydrate levels but high fat and protein amounts. The principal purpose of the protein in the diet is to allow optimal maintenance of body protein (lean body mass), and the purpose of the fat is to provide sufficient energy to accommodate this protein sparing effect while allowing the development of mild ketosis. The diet has a fat portion that supplies 50-95%, preferably 50-80%, of the calories; a protein portion that supplies 5-40%, preferably 10-35%, of the calories; and if there is any carbohydrate in the diet, it is limited to 0-10% of the calories. This is in contrast to traditional diets that have 45-65% of the calories from carbohydrates. One of the important aspects of the diet is the choice of the fat content. Preferably, at least the majority of the fat calories are supplied by an oil rich in DHA and EPA, with medium chain triglycerides (MCT's), monounsaturated fats and oils rich in n-9 fats such as oleic acid being another prime source of calories. In contrast to most diets that use high fat, the amount of linoleic acid is limited to 0-3%, more preferably 0-<1%, of the fat calories. When fish oil high in DHA and EPA provide at least 15% of total energy expenditure, added linoleic acid need not be present, with small amounts of linoleic acid (up to 1%) required when fish oil is not present. As approaching energy balance and expenditure is particularly important to critically ill patients, the ketogenic diet should provide 50-90% of the caloric needs in terms of energy expenditure of the critically ill patient. There need not be any additional fat or protein provided to the patient (and carbohydrate often found in normal IV solutions should be avoided), and it is expected that at least for the short time that a critically ill patient is often on the ketogenic diet, energy balance will generally not be achieved. In addition, the diet normally contains at least 0.5% (by calorie) arachidonic acid, which is provided when fish oil is given in sufficient amounts. In addition, the diet preferably contains all essential micronutrients such as vitamins and minerals.

The diet may be delivered in several different formats, with modifications made as needed depending on the format chosen. For most critically ill patients, the diet may be in the form of enteral nutrition as that is how most of these patients are currently fed. These formulations would likely have little or no carbohydrate, since most critically ill patients have a fluid IV that normally includes glucose in the solution. This improved enteral formulation could replace classic formulations for those critical care patients fed by tube. The advantages of using the present formulation include lower volume for the same caloric input (because of the higher caloric content of fats versus carbohydrates), less triggering of insulin production and a lower formation of oxidative reactant species. As noted, MCT's may constitute a percentage of the fat calories since they are absorbed easily by the body and are preferred ketogenic precursors. In addition, the proper selection of fats in the formulation can provide anti-inflammatory or anti-infection advantages.

However, many critically ill patients cannot receive sufficient calories by enteral nutrition. Some patients have trouble digesting enteral nutrition for a variety of reasons. For these patients, parenteral nutrition (feeding through an intravenous tube) is required. The present formulations can be modified to a parenteral nutrition form to treat these patients. For certain patients, parenteral nutrition is provided through a central vein and the formulation may be used as total (or partial) parenteral nutrition. In other cases, the formulation may be fed through a peripheral vein as total (or partial) peripheral nutrition. Because fat formulations are calorically dense and fat would provide the bulk of the calories, full feeding is possible through total peripheral nutrition which is not possible with a carbohydrate-based system. In either case, because this formulation is going directly into the blood stream without passing through the stomach or intestine, particle size and osmolality must be considered. In addition to fish oil, which has about 10% lipid and provides about 1 kilocalorie/cc, there are presently available 20% and 30% lipid emulsions, and these provide respectively 2 and 3 kilocalories/cc which could provide most of the daily caloric content in 1 liter or less. The formulation can be pre-mixed in a bag, with separate chambers for the fat and protein, or the different parts of the formulation can be provided for contemporaneous mixing.

The invention also features a method of providing nutritional support to a critically ill patient by administering a ketogenic diet to the patient. The ketogenic diet used in these circumstances is as described above; that is, it should contain about 50-95%, preferably 50-80%, of the calories in the form of fat, 5-40%, preferably 10-35%, of the calories in the form of protein, and 0-10% of the calories of the form of carbohydrate. The fat content can preferably be 95%, 90%, 85%, 80%, 75%, 70%, or 65% of the diet. The majority of the fat in the diet is fat is selected from the group consisting of oils rich in DHA and EPA, concentrated omega-3 rich oils, MCT's, and n-9 rich oils such as oleic acid. The linoleic acid content of the diet should provide less than 3%, preferably less than 1%, of the total calories. The diet should provide 50-90% of the energy expenditure of the patient, with no other energy sources being given. The inventive nutritional support can be provided through enteral or parenteral feeding.

### DETAILED DESCRIPTION

The present invention relates to ketogenic diets and their use in the treatment of the critically ill patient. The use of the ketogenic diet for treating the critically ill is counterintuitive as glucose-rich diets have been recommended and used for years. The use of glucose-rich diets was chosen because of the high energy yield from this substrate in terms of oxygen efficiency and the role of glucose as the preferred fuel for the macrophages and fibroblasts, key cell types in the systemic inflammatory response when oxygen levels are limited. However, the classic glucose-based diet may lead to problems with fluid retention, the production of oxidative radicals, and hyperglycemia. In addition, sepsis problems are increased in the critically ill. Furthermore, additional glucose in the diet is not needed as there is ample glucose from endogenous production during the severe systemic inflammatory response to meet the needs of macrophages for their bactericidal actions and fibroblasts for connective tissue repair. The ketogenic diet of the present invention provides sufficient energy while ameliorating these problems. The use of omega-three (“n-3”) rich fats and MCT's as the primary caloric energy source provides anti-inflammatory benefits on the one hand (primarily from the “n-3's”) and an optimal ketogenic substrate (MCT's) in addition to lower fluid loads than conventional diets. In addition, since the carbohydrate level is kept very low, harmful levels of hyperglycemia are ameliorated or prevented and the antidiuretic effects of insulin are reduced as well. Finally, a ketogenic diet increases the redox potential, which increases the antioxidant potential of the diet, particularly by increasing the ratio of reduced to oxidized glutathione, enabling the body to better deal with oxidative stress.

### Definitions

The following definitions will be used throughout:

“Ketogenic diet” means a high fat, adequate protein, low carbohydrate diet.

“DHA” means docosahexaenoic acid.

“EPA” means eicosapentaenoic acid.

“MCT” means medium chain triglycerides.

“Critically ill” means patients having an Acute Physiology and Chronic Health Evaluation (“APACHE”) score of 15 or higher.

“Oil rich in DHA and EPA” means an oil having approximately 150-550 mg of EPA and approximately 250-500 mg DHA in 0.5-1.5 g of oil.

“Essential micronutrients” means vitamins, minerals, and other essential nutrients as described in the initial Dietary References Intake (DRI) of the United States.

“High oleic acid containing oils” are oils having about 50% or more of the triglycerides containing oleic acid.

### DESCRIPTION

The ketogenic diet of the present invention is a dramatic change in the treatment of critically ill patients. The classic diet for treatment of the critically ill was heavily carbohydrate-based as that was thought to provide a better energy source. The use of this high carbohydrate diet has led to the common problems of fluid overload, excessive oxidative radicals, and hyperglycemia, each of which may worsen clinical outcome. While ketogenic diets have been used for certain patients, they have not been used for the critically ill.

Preferably, a diet for the critically ill will provide 1.5 g/kg body weight of a mixture of protein, supplemented by essential vitamins, nutrients and minerals. The critically ill diet should provide at least 50% of the energy requirements of the patient with fat and protein primarily meeting the caloric goals and limited carbohydrate, with greater amounts of calories being provided as tolerated, with the goal of meeting total energy expenditure. However, the critically ill normally often cannot obtain this latter level of caloric intake, primarily because of various issues associated with enteral feeding. Three of the major adverse consequences of classic full nutritional support of the critically ill with a carbohydrate-based feeding regimen are hyperglycemia, volume overload and excessive oxidative stress. Each of these is associated with worsening outcome for the critically ill and is exacerbated by aggressive feeding.

Hyperglycemia often occurs in the critically ill because the systemic inflammatory response increases the rate of glucose production by up to 50%. This same inflammatory response makes gluconeogenesis less responsive to the provision of exogenous glucose and limits peripheral glucose uptake due to insulin resistance. Thus, providing carbohydrate as a major feeding source leads to increases in glucose appearance and decreased clearance, often leading to pathological hyperglycemia in the critically ill. The iatrogenic production of hyperglycemia, particularly when raised to levels greater than 180-200 mg/dl, increases the risk of nosocomial infection.

Volume overload commonly occurs because increases in fluid are needed to provide the relevant nutrition while the organ dysfunction and hormonal changes in the critically ill reduce the ability to excrete fluid. Hormonal secretions affecting fluid and electrolyte balance include increases in the antidiuretic hormones, aldosterone and insulin, that foster excessive sodium and water retention and thus volume overload.

The systematic inflammatory response also includes an increase in the production of reactive oxygen species that damage and impair the function of normal tissue. Hyperglycemia increases cytokine production and the production of reactive oxygen species as well. With parenteral glucose administration, there is a marked increase in de novo lipogenesis with more than 50% of the administered glucose initially being metabolized by non-oxidative pathways, primarily de novo lipogenesis, which lowers the cytosolic and mitochondrial redox state. This alteration in the redox state also reduces the ratio of reduced to oxidized glutathione, which reduces the antioxidant capacity to handle the oxidative stress of inflammation and infection. In addition, gluconeogenesis is increased and not suppressed by the exogenous glucose, which when combined with glucose being fed and impaired uptake of glucose by skeletal muscle, leads to hyperglycemia of a magnitude to adversely impact organ function and clinical outcome.

The diets of the present invention help to treat these problems. The use of high fat and low glucose assists in preventing hyperglycemia. Because of the higher caloric content of fat versus carbohydrate, lower fluid volumes are needed, ameliorating fluid overload. The particular fats selected, primarily oils rich in DHA and EPA, help reduce systemic inflammation, thus improving many of the associated problems. The reduction in de novo lipogenesis and gluconeogenesis improves the cellular redox state which assists in neutralizing reactive oxygen species. The use of MCT as a portion of the fat decreases inflammation and fosters ketogenesis.

The diets of the invention are particularly useful as a replacement for the present high carbohydrate enteral formulations. Complete formulas for providing energy via tube (enteral nutrition) are intended to provide 50%-120% of the energy expenditure. For the critically ill patients, enteral feeding normally provides 50-75% of the energy expenditure as the patient normally cannot tolerate 100% of the calories required due to intestinal dysfunction. Levels higher than this are normally used for the recuperative phase of the critical illness or for use in neurological illnesses such as Alzheimer's, post-stroke, epilepsy, other senile dementias, and the recuperative phase of brain injury. To provide the desired ketogenic effect, the diet should have less than 100 g carbohydrate per day, preferably having only 0-3% of the caloric total in the form of carbohydrate. Any carbohydrates in the diet should preferably have either low glycemic index or low insulinogenic properties with the intent of keeping ketonemia (beta hydroxybutyrate) levels at least 0.05-4 mM with optimal levels in the 0.25-2 mM range.

The present diet must provide protein as well as fat. Normally, the protein should be least 1 g/kg/day if greater than 75% of the energy expenditures is provided. At lower levels of providing energy expenditure through the diet, such as those used in critically ill patients, at least 1.2 g/kg/day, preferably 1.5 g/kg/day or more, of protein should be provided. Protein sources of high biologic value such as casein, whey, soy or the like are preferable but complementary proteins from vegetable sources may be used. Whole proteins are preferred but mixtures of amino acids to provide all of the essential amino acids and a mixture including non-essential amino acids may be used as either free amino acids or small peptides. The critically ill formula should also allow for supplementation of important amino acids that may provide unique benefits in large quantities, such as branched-chain amino acids which normally provide about 20% of the total protein but could be as much 50% with leucine in iso-molar quantities with isoleucine and valine. Glutamine, arginine and sulfur containing amino acids such as methionine and cysteine may be used in higher quantities compared to the amounts in the normal protein in the diet. For example, the levels of methionine and cysteine can up to three times that found in high biologic value animal protein. By selecting these amino acids judiciously, optimization of the protein intake and catabolism is achieved.

The type of fat content of any ketogenic diet, but with this diet in particular, is important.

Because this diet is low in carbohydrates, the fat portion will provide at least 50-80% of the calories for energy expenditure. The preferred formulation would be an oil rich in DHA/EPA such as fish oil from high EPA/DHA containing species and medium chain triglycerides (MCT), with the range of each being 20-80% of the fat calories. Separate DHA or EPA may be used but normally high EPA/DHA oils form the basis of the diet. Other fats, such as those with a high oleic acid content but low linoleic acid content, such as olive oil or pecan oil, may be employed. A 1:1:1 ratio of fish oil, MCT, and high oleic acid-containing oil would be preferred, but the optimal formulation would always have substantial and approximately equal amounts of fish oil and MCT's. The fish oil should to be a least 20% of the total calories to meet essential fatty acid requirements if used as the sole source of long chain fat in the diet. The EPA/DHA component will be anti-inflammatory with neuroprotective and regenerative effects. The MCT is known to provide ketogenesis and is normally used in many ketogenic diets. In lieu of the fish oil, a formulation of DHA/arachidonic acid (AA) can be used as the source of EPA/DHA, since these two fatty acids can meet essential fatty acid needs with one third of the amount of fat necessary as when provided as fish oil. If using this DHA/AA formulation, at least 7% of the total energy must be met with this in a 80:20 fat:protein diet, with the optional ratio of 25:75 DHA/AA:MCT. The formulation also allows for the use of omega 6 (n-6) rich oils, in particular vegetable oils such as soybean, safflower or corn oil but due to their high linoleic acid content which can be proinflammatory, high oleic acid-oils such as olive oil are preferred and can be used in mixtures with the fish oil or DHA/MCT, usually in a 50:50 mixture along with MCT. For example, a fat component of the diet, having 25% fish oil, 25% vegetable oil (olive oil) and 50% MCT, could be used to provide the benefit of the diet. No other long chain fatty acids are required if the fish oil makes up a least 15% of the calorie content of the diet.

The diet should contain the recommended dietary amount of all vitamins, minerals and other essential nutrients per the Dietary Reference Intake. This includes at least 20 mEq per day of sodium, with 70-100 mEq preferable, and 20-100 mEq per day of potassium with 40-80 mEq per day preferable. There should also be supplementation of certain antioxidant essential nutrients such as zinc, selenium, vitamin A, vitamin C and vitamin D in amounts up to 5 times the Recommended Dietary Allowance. Other nonessential nutrients such as carnitine, creatine and carnosine may be included.

The ketogenic diet of the present invention may also be used for parenteral nutrition, either through peripheral veins or through the central vein. If the formulation is being used through a peripheral vein, protein is best provided in the form of amino acids that are isotonic or only slightly hypertonic (3-5%). The diet can include as the fat source only fish oil, fish oil: MCT in 25-75% quantities of each, fish oil: MCT: n-6 long chain triglycerides (LCT) in 25-75% quantities, or MCT: LCT in 25-75% quantities of each. MCT amounts should be limited to no more than 75% of the total fat. Any carbohydrate should be limited to that provided outside the feeding regimen for the provision of antibiotics and other medication and the total should be less than 100 g/24 hours from all sources, preferably less than 85 g/24 hours, taking note that lipid emulsions contain about 22.5-25 g of glycerol/liter which should be included in this calculation.-Electrolytes, minerals and vitamins would be the same as is used in an enteral formulation, except tonicity with electrolytes is an issue in parenteral formulations provided through peripheral veins. In the central vein parenteral formulation, the composition would be substantially the same but the protein level in composition could be higher, with 4.5-15% amino acids, because the central vein can tolerate the higher tonicity and/or osmolality.

The ketogenic diet should provide 50-100% of the predicted energy expenditure and should prevent the development of a blood glucose level exceeding 180-200 mg/dl in non-diabetic patients as well as many with type 2 diabetes due to the near absence of dietary carbohydrate. This diet should allow for excellent blood glucose homeostasis and the attendant reduction of nosocomial infection without the risk for intensive insulin treatment, although insulin in modest amounts may be necessary in the most severely ill or in those with type 2 diabetes. This dietary regimen is contraindicated in Type 1 diabetes due to the greater concern about preventing hypoglycemia when administering insulin to such patients. Gluconeogenesis would be reduced compared with conventional diet as much of the brain's energy is being met by ketone bodies and with the muscle completely dependent on fatty acids and some ketone bodies as fuel. De novo lipogenesis would be dramatically ameliorated by the limitation of the dietary carbohydrate. The dramatic reduction in both forms of reductive biosynthesis, gluconeogenesis and de novo lipogenesis, confirmed by the high β-hydroxybutyrate/acetoacetate ratio, should support the heightened function of antioxidants, particularly the glutathione system to reduce oxidative stress. This is likely to improve organ function and provide a beneficial clinical outcome. The ketogenic diet will also lead to diuresis in part due to the fall in insulin levels and in part to the urinary excretion of ketone bodies which require a cation, usually sodium and potassium to accompany them. Insulin is a potent antinatriuretic and antidiuretic hormone through its renal effects. Thus, fluid imbalances should be easier to manage and prevent. Because of the higher caloric content of the fat as compared to glucose and the much lesser osmolality of fat for peripheral vein feeding, the total amount of liquid to provide the same caloric content is less, which will provide fewer problems for the management of fluid balance. However the high-fat content of the enteral ketogenic diet will slow gastric emptying which will need to be monitored. However, the diet will also have a lower osmotic load due to the near total lack of low molecular weight carbohydrate, which should improve gastric emptying. It is likely that modest degrees of hypocaloric feeding (50-90% of the energy requirements) may be optimal when feeding into the stomach, although larger amounts should be well-tolerated in post-pyloric or jejunal feeding. An additional benefit of such a high-fat formula in the critically ill could be the stimulation of the cholinergic anti-inflammatory pathway by fat.

Although enteral feeding and enteral diets would be the most likely used for the ketogenic diet, the diet may be used as an exclusively parenteral diet or as a supplement to an enteral diet. If used as a supplement to an enteral diet, isotonic or mildly hypertonic amino acids (3%-4.5%) and isotonic fat emulsions (10%-30%) should be used, with only the latter containing small amounts of carbohydrate (e.g., 22.5-25 g of glycerol per liter). If used as a parenteral solution administered through the central vein, higher amino acid content (4.5%-14%) is possible, because this form of administration allows the use of highly hypertonic solutions. The volume may be reduced in this way.

There were concerns with past ketogenic diets about the lack of fiber making the diets unsuitable for long term use. For the critically ill, this is not a major problem, since the diet is likely only to be used for a limited time. For longer term use, however, it is possible, to add fermentable fibers to the diet without changing the ketogenic properties of the diet, although technically carbohydrates, because fermentable fibers are not anti-ketogenic. Ketonemia does lead to elevation of serum uric acid for the first several weeks by competing with ketone bodies for urinary excretion, which could theoretically lead to gout but it is unlikely to lead to uric acid renal stones. But this is a very rare complication. With the adaption to chronic ketonemia, the preferential urinary excretion of ketone bodies is reversed over number of weeks. The other known problems with ketogenic diet, increased satiety and in children, reduced growth rate, are not issues for short-term use of these diets in the critically ill.

### Example 1

This example provides an exemplary enteral formulation for treating the critically ill. The formulation is intended to provide 50-75% of the predicted energy expenditure of the critically ill patient.

Normal energy expenditure for a critically ill 70 kg male would be about 25-30 kilocalories/kg or approximately 1700-2000 kilocalories/day. The diet provides 1300-1500 kilocalories/day, with 100 grams of protein as whole protein containing a 50:50 mixture of casein (whey) and soy. Fat would be 100 g, 50 g as fish oil and 50 g as MCT provided in 1000 cc of enteral formula containing 100 g of protein and 100 g of fat with water to make up the rest of the emulsion. All essential nutrients except linoleic acid (including minerals and vitamins) would be provided in RDA amounts. Sodium would be provided as 70 milliequivalents (meqs) of sodium chloride, potassium as 60 meqs potassium acetate, with 40 millimoles of phosphorus as equal amounts of the sodium and potassium phosphate salts.

Dietary fiber is normally not needed in the diet because the treatment is normally for a short period. However, if the patient needs fiber in the diet because of constipation or the diet is to be used for a sustained period, fermentable fiber can be added as it not anti-ketogenic.

### Example 2

This example provides an exemplary parenteral formulation for treating the critically ill. The formulation is intended to provide 50-75% of the predicted energy expenditure of the critically ill patient. Protein is provided as an amino acid mixture in amounts of 90 g that would be 40% essential, 60% non-essential amino acids providing adequate amounts of each essential amino acids in 1500 cc with amino acids (6%) and 4% fish oil, 4% MCT for parenteral administration. Glycerol would be provided as 25 g. All essential vitamins and minerals would be provided in RDA amounts. In addition 90 meq sodium, 60 meq potassium, 70 meqs chloride, 40 meqs acetate and 40 meqs phosphate will be provided as sodium chloride, sodium phosphate, potassium acetate and potassium phosphate. This can be formulated in the individual pharmacy using base solutions of 10% amino acids, and 20% fish oil/MCT mixture or individual formulations of 20% fish oil and 20% MCT. This could also be a ready to mix container with 1000 cc of 10% amino acids and 600 cc of a 20% fish oil/MCT mixture in separate chambers that can be mixed before use.

### Example 3

This example describes a model for testing of the ketogenic diet in treating the critically ill. The sepsis model, where mice are treated with lipopolysaccharide (“LPS”), is used to show an initial hyperglycemic response followed by a hypoglycemic response. The test shows the benefit of a ketogenic diet in normalizing glucose levels and reducing inflammatory response.

C57/B16 mice were fed either a ketogenic diet (KD) with 90% kcal from fat, 0.4% kcal from carbohydrate, and 9.1% kcal from protein, or a control diet (CD) with 2.5% kcal from fat, 77.4% kcal from carbohydrate, and 10% kcal from protein for 4 weeks. After 4 weeks, half of the mice from each diet group received 10 mg/kg of intraperitoneal LPS and half received an equivalent volume of saline (n=10 per group). Fasting blood glucose was tested at 15, 30, 60, 90, 120, 180, and 240 min. After 4 hours, animals were sacrificed and serum was analyzed for levels of insulin, IL-6, and TNF-a. Liver sections were stained with hematoxylin and eosin, and periodic acid-Schiff stain, and analyzed by a pathologist. Quantitative PCR was used to determine transcription levels of NFkB to characterize downstream inflammatory signaling.

Results:

Both KD and CD mice demonstrated an initial hyperglycemic response to LPS injection, while KD and CD mice maintained normal serum glucose after saline injection. Hypoglycemia (glucose<80 mg/dL) developed 3 hours after LPS injection in only the CD mice, whereas KD mice normalized their blood glucose. By 4 hours after LPS injection, mean serum glucose was 47.8 mg/dL in the CD mice and 92 mg/dL in the KD mice (p=0.0001). In addition, HOMA-IR (measure of insulin resistance) was 10 times higher in KD mice (0.9), compared to CD mice after LPS (0.09) (p=0.01). Glycogen content was undetectable within liver specimens from CD mice after LPS, while glycogen stores were still present in livers from KD mice after LPS. Liver sections were otherwise normal in all groups. IL-6 levels were not significantly different between the groups, however TNF-a was elevated in the KD mice (841 pg/mL) compared to CD mice (534 pg/mL, p=0.03) after LPS. Notably, a 10-fold increase in NFkB mRNA transcription was found within livers of CD mice after LPS, while a 5-fold increase was noted in livers of KD mice compared to CD mice after saline (p=0.001).

What is claimed is:

1. A ketogenic diet for treating critically ill patients having at least a fat portion and a protein portion, the diet comprising:
a fat portion that supplies 50-95% of the calories; and
a protein portion that supplies 5-35% of the calories; and, wherein any carbohydrate in the diet is limited to 0-10% of the calories.

2. The ketogenic diet of claim 1 wherein:
at least the majority of said fat calories are supplied by an oil rich in DHA and EPA.

3. The ketogenic diet of claim 1 wherein:
the fat portion comprises linoleic acid, wherein the linoleic acid content of the fat portion is limited to less than 3% of said fat calories.

4. The ketogenic diet of claim 3 wherein said diet contains less than 1%, by calorie, linoleic acid.

5. The ketogenic diet of claim 1 wherein said ketogenic diet provides 50-90% of the caloric needs of the critically ill patient.

6. The ketogenic diet of claim 1 wherein the majority of said fat calories are supplied by fats selected from the group consisting of medium chain triglycerides, high oleic acid containing oils and saturated fats.

7. The ketogenic diet of claim 6 wherein said diet contains at least 0.5% by calorie arachidonic acid.

8. The ketogenic diet of claims 7 wherein said diet contains less than 1%, by calorie linoleic acid.

9. The ketogenic diet of claims 1 further comprising all essential micronutrients.

10. The ketogenic diet of claim 1 wherein said diet is in the form of enteral nutrition.

11. The ketogenic diet of claim 1 wherein said diet is in the form of parenteral nutrition.

12. The ketogenic diet of claim 1 wherein said diet comprises 50-80% of the calories in the form of fat, 10-35% of the calories in the form of protein, and 0-10% of the calories of the form of carbohydrate.

13. A method of providing nutritional support to a critically ill patient comprising:
administering a ketogenic diet to said patient and limiting the carbohydrate to less than 10% of the total calories given to said patient.

14. The method of claim 13 wherein said ketogenic diet comprises 50-95% of the calories in the form of fat, 5-40% of the calories in the form of protein, and 0-10% of the calories of the form of carbohydrate.

15. The method of claim 14 wherein said ketogenic diet comprises 50-80% of the calories in the form of fat, 10-35% of the calories in the form of protein, and 0-10% of the calories in the form of carbohydrate.

16. The method of claim 14 wherein the majority of the fat in the diet is a fat is selected from the group consisting of oils rich in DHA and EPA, concentrated omega-3 rich oils, MCT's and high oleic acid containing oils.

17. The method of claim 14 wherein the linoleic acid content of the diet is less than 3% of the total caloric intake.

18. The method of claim 17 wherein the linoleic acid content of the diet is less than 1% of the total caloric intake.

19. The method of claim 13 wherein the ketogenic diet provides 50-90% of the energy expenditure of the patient.

20. The method of claim 13 wherein said ketogenic diet is in the form of enteral nutrition.

21. The method of claim 13 wherein said ketogenic diet is in the form of parenteral nutrition.

22. The method of claim 13 wherein the carbohydrate in the diet is less than 100 g/24 hours.

23. The method of claim 22 wherein the carbohydrate in the diet is less than 85 g/24 hours.
